(19) United States
(12) Patent Application Publication
(10) Pub. No.: US 2007/0055134 A1
(43) Pub. Date: Mar. 8, 2007

(54) Undersampled magnetic resonance imaging

(76) Inventor: Miha Fuderer, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/555,574

(22) PCT Filed: May 4, 2004
(86) PCT No.: PCT/IB04/50572

§ 371 Date: Nov. 4, 2005

(30) Foreign Application Priority Data
May 6, 2003 (EP) 03101253.7

(51) Int. Cl. A61B 5/05

(52) U.S. Cl. 600/410; 600/420

(57) ABSTRACT

A novel magnetic resonance imaging method is described for forming a sequence of images from a plurality of signals acquired by at least one receiver antenna. Each receiver antenna has a spatial sensitivity profile. An activity map is calculated as a standard deviation over a series of images acquired by a reference scan. Thereupon, the object is sampled in an actual scan in an interleaved manner in k-space with a reduction factor. The resulting data is Fourier transformed to the spatial domain to form a sequence of folded preliminary images, and the fold-over artefacts or the ambiguity in the preliminary images resulting from the undersampled data in k-space is resolved in forming the actual images on the basis of the activity map.

The invention relates to a magnetic resonance method for forming a sequence of images from a plurality of signals acquired by at least one receiver antenna according to the preamble of claim 1. The invention also relates to a magnetic resonance imaging apparatus for obtaining an image according to the preamble of claim 5 and to a computer program product according to the preamble of claim 8.

In magnetic resonance imaging there is a general tendency to obtain acceptable images within shorter periods of time. For this reason the sensitivity encoding method called “SENSE” has recently been developed by the Institute of Biomedical Engineering and Medical Informations, University and ETH Zürich, Switzerland. The SENSE method is based on an algorithm which acts directly on the image as detected by the coils of the magnetic resonance apparatus and which subsequent encoding steps can be skipped and hence an acceleration of the signal acquisition for imaging by a factor of from two to three can be obtained. Crucial for the SENSE method is the knowledge of the sensitivity of the coils which are arranged in so called sensitivity maps. In order to accelerate this method there are proposals to use raw sensitivity maps which can be obtained through division by either the “sum-of-squares” of the single coil references or by an optional body coil reference (see e.g. K. Pruessmann et. al. in Proc. ISMRM, 1998, abstracts pp. 579, 799, 803 and 2087). In fact the SENSE method allows for a decrease in scan time by deliberately undersampling k-space, i.e. deliberately selecting a Field-of-View (FOV) that is smaller than the object to be acquired. From this undersampling fold-over artefacts are obtained which can be resolved or unfolded by the use of the knowledge of a set of distinct coils having different coil sensitivity patterns. The undersampling can be in either one of both phase-encoding directions.

The SENSE method is preferred for acceleration of the signal acquisition for magnetic resonance imaging resulting in an enormous reduction in operating time. However, the method can only be used properly if the coil sensitivity is exactly known. Otherwise imperfections will cause fold-over artefacts (aliasing) which lead to incorrect images. In practice the coil sensitivity cannot be estimated perfectly and will be dependent on fluctuations in time (movement of the patient, temperature influences, etc.).

Another important problem of the SENSE method is the spatially varying noise level in the resultant image. More specifically, the resultant image can have regions of extremely high noise level that are due to local “underdetermination” of the information provided by the coil patterns.

Another kind of undersampling may be applied in dynamic imaging as has been described in T. J. Provost, SMRI 1990, Works-in-progress, abstract 462. If a part of the object is known to be static, advantage can be taken from this knowledge. In the simplest case, where exactly one half of the FOV is known to be static, k-space density can be reduced to a factor of 2. This results in folding of image data. However, exactly one pixel of the dynamic object area overlaps with exactly one pixel of a static area. If, in whatever way the static image is known, the static aliasing can be subtracted from the required dynamic image part. That static image can be measured beforehand, afterwards, or by shifting k-space rows from one frame to the other, in order to reconstruct a non-aliased (but temporally blurred) image (see e.g. Madore, Glover and Pelc, MRM 42. p. 813-828 (1999)).

It has further been proposed in U.S. Pat. No. 6,353,752 to convert the knowledge that some part of the FOV is static or not very dynamic into an increase in temporal resolution for the dynamic part, or into a reduction in the scan time. It is shown that if only 1/n of the FOV is dynamic, only 1/n of the k-space portions need to be acquired multiple times. The remaining fraction (n−1)/n of the k-space portions can be acquired only once, leading to an increase by about a factor n in temporal resolution or a decrease by about n in scan time. It is not mentioned in which manner the static part of the image is revealed.

On the other hand, there is a proposal to combine SENSE with spatial or temporal filtering (P. Kellman et. al. Adaptive Sensitivity Encoding Incorporating Temporal Filtering (TSENSE) ISMRM 45: p. 846-852, 2001).

All of the above mentioned methods have in common that part of the acquired region is to be considered as “stationary” while another part is not. Some of the methods take for granted that only the most central half is moving (e.g. U.S. Pat. No. 6,353,752), others require some input of the user on stationarity (although quite elementary).

It is an object of the present invention to achieve a further acceleration of imaging of the above mentioned SENSE method while the quality of the obtained images is remained.

This and other objects of the invention are achieved by a method as defined in claim 1, by an apparatus as defined in claim 5 and by a computer program product as defined in claim 8.

The main aspect of the present invention is based on the idea that an acceleration e.g. by the SENSE method is not only feasible by increasing the number of recording coils but also by making use of the intrinsic knowledge of the activity profile of the object to be imaged. Each receiver antenna has a spatial sensitivity profile. An activity map is calculated as a standard deviation over a series of images acquired by a reference scan. Thereupon, the object is sampled in an actual scan in an interleaved manner in k-space with a reduction factor. The reduction factor represents the amount of undersampling of the magnetic resonance signals relative to a full sampling of k-space required in view of a predetermined spatial resolution of the magnetic resonance image. The resulting data is Fourier transformed to the spatial domain to form a sequence of folded preliminary images, and the fold-over artefacts or the ambiguity in the preliminary images resulting from the undersampled data in k-space is resolved in forming the actual images on the basis of the activity map.

These and other advantages of the invention are disclosed in the dependent claims and in the following description in which an exemplified embodiment of the invention is described with respect to the accompanying drawings. Therein shows:

FIG. 1 a sequence of images acquired in a pre-scan,

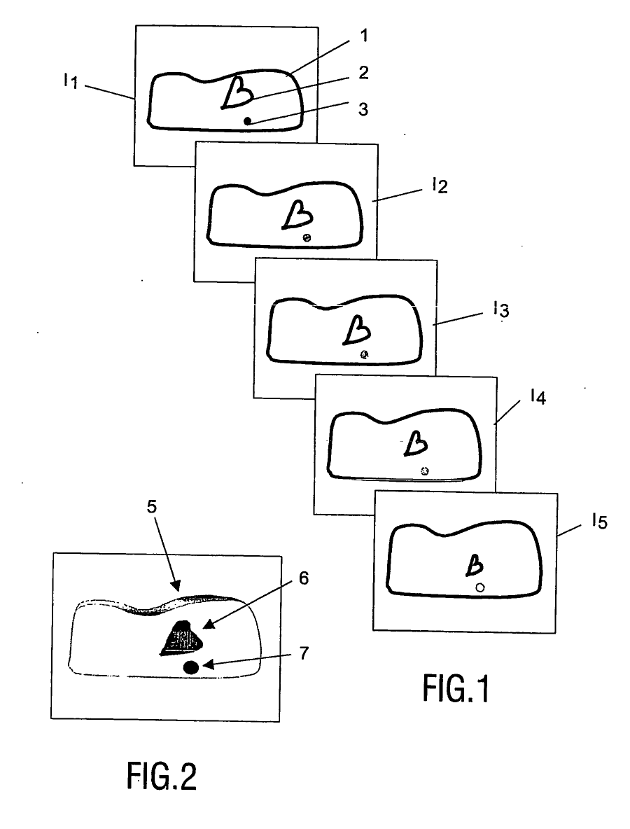

FIG. 2 a diagram of the activity profile extracted from the sequence of FIG. 1,

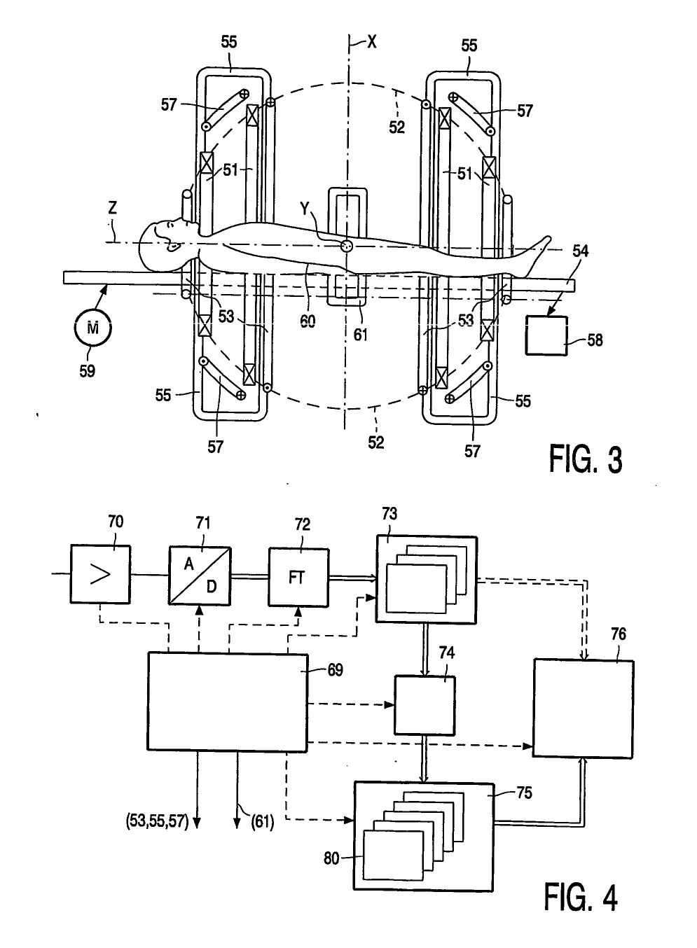

FIG. 3 an apparatus for carrying out the method in accordance with the present invention, and

FIG. 4 a circuit diagram of the apparatus as shown in FIG. 3.

The here described method applies to dynamic MRI sequences, whether in a cartesian or non-cartesian frame (like radial or spiral). It is assumed that at least a part of an object under study has interesting temporal frequencies of change up to f/2, which means that a frame has to be acquired every T_{D}=1/f seconds. The object as a whole has a size of the Field-of-View (FOV), which would dictate a k-space step or density in case of non-cartesian scans of no more than Δk. Whereas it is assumed that no acceleration techniques are used.

The region to be imaged, whether a 2D slice or a 3D volume, is segmented into regions of “distinct activity” (as opposed to the known “stationarity”). Therefore, an “activity map” of the considered object will be obtained in a separate scan, e.g. in a calibration measurement before the actual scan. For an average low resolution 3D scan the acquisition of body-coil signal and synergy-coil signals are interleaved. Then the body-coil signals and the synergy-coil signals are transformed into specific volumes, and the division of both volumes gives an estimate of the coil sensitivity for each location. From each average scan it is relatively easy to generate a body-coil volume and to calculate a standard-deviation map: a deviation over a time period (i.e. over the average scans) for each location in the volume. These data are readily usable as an indication of local activity of the object to be imaged and form the basis of the activity map. The so formed activity map is now implemented in that the acquisition of the “activity knowledge” is integrated with the acquisition of coil-sensitivity calibration data which is used for unfolding according to the SENSE method.

The acquisition sequence of the present method has the following characteristics:

1. A series of signals are acquired by at least one receiver antenna for recording signals. Each receiver antenna has a spatial sensitivity profile. That is, the senstivity value of the receiver antenna for magnetic resonance signals depends on the location where the magnetic resonance signals origingate from relative to the receiver antennae. These sensitivity values as a function of positions form the spatial sensitvity profile of the receiver antenna.

2. From the series of images, which are obtained by the pre-scan, an activity map is calculated as a standard deviation over the images.

3. The object is then sampled in an actual scan in an interleaved manner in k-space with a predetermined reduction factor.

4. Thereupon the resulting data is Fourier transformed to the spatial domain, in order to form a sequence of folded preliminary images.

5. From the preliminary images, resulting from the undersampled data in k-space, the fold-over artefacts or in general terms the ambiguity is resolved in that a series of actual images is formed on the basis of the activity map.

As an example in FIG. 1 the above described method is applied to dynamic 2D imaging of a part of the body 1 in a cross-section through the heart 2 (fast motion) and the spine 3 (no or slow motion). In a sequence of images I₁ to I₅ the motion of the heart 2 and of the spine 3 is schematically depicted. From this information an activity profile can be obtained in the manner as described above, which is shown in FIG. 2. As can be seen the activity of the body 5 is more in the region of the belly, and the activity of the heart 6 is much larger than the activity of the spine 7.

Although the method is most effective with SENSE, it can also be used without any parallel imaging like SENSE. In practice the reduction factor is normally an integer or non-integer number greater than 1.

The apparatus shown in FIG. 3 is an MR apparatus which comprises a system of four coils 51 for generating a steady, uniform magnetic field whose strength is of the order of magnitude of from some tenths of Tesla to some Tesla. The coils 51, being concentrically arranged relative to the z axis, may be provided on a spherical surface 52. The patient 60 to be examined is arranged on a table 54 which is positioned inside these coils. In order to produce a magnetic field which extends in the z direction and linearly varies in this direction (which field is also referred to hereinafter as the gradient field), four coils 53 as multiple receiver antennae are provided on the spherical surface 52. Also present are four coils 57 which generate a gradient field which also extends (vertically) in the x direction. A magnetic gradient field extending in the z direction and having a gradient in the y direction (perpendicularly to the plane of the drawing) is generated by four coils 55 which may be identical to the coils 57 but are arranged so as to be offset 90° in space with respect thereto. Only two of these four coils are shown here.

Because each of the three coil systems 53, 55, and 57 for generating the magnetic gradient fields is symmetrically arranged relative to the spherical surface, the field strength at the centre of the sphere is determined exclusively by the steady, uniform magnetic field of the coil 51. Also provided is an RF coil 61 which generates an essentially uniform RF magnetic field which extends perpendicularly to the direction of the steady, uniform magnetic field (i.e. perpendicularly to the z direction). The RF coil receives an RF modulated current from an RF generator during each RF pulse The RF coil 61 can also be used for receiving the spin resonance signals generated in the examination zone.

As is shown in FIG. 4 the MR signals received in the MR apparatus are amplified by a unit 70 and transposed in the baseband. The analog signal thus obtained is converted into a sequence of digital values by an analog-to-digital converter 71. The analog-to-digital converter 71 is controlled by a control unit 69 so that it generates digital data words only during the read-out phase. The analog-to-digital converter 71 is succeeded by a Fourier transformation unit 72 which performs a one-dimensional Fourier transformation over the sequence of sampling values obtained by digitization of an MR signal, execution being so fast that the Fourier transformation is terminated before the next MR signal is received.

The raw data thus produced by Fourier transformation is written into a memory 73 whose storage capacity suffices for the storage of several sets of raw data. From these sets of raw data a composition unit 74 generates a composite image in the described manner; this composite image is stored in a memory 75 whose storage capacity suffices for the storage of a large number of successive composite images 80. These sets of data are calculated for different instants, the spacing of which is preferably small in comparison with the measurement period required for the acquisition of a set of data. A reconstruction unit 76, performing a composition of the successive images, produces MR images from the sets of data thus acquired, said MR images being stored. The MR images represent the examination zone at the predetermined instants. The series of the MR images thus obtained from the data suitably reproduces the dynamic processes in the examination zone.

The units 70 to 76 are controlled by the control unit 69. As denoted by the downwards pointing arrows, the control unit also imposes the variation in time of the currents in the gradient coil systems 53, 55 and 57 as well as the central frequency, the bandwidth and the envelope of the RF pulses generated by the RF coil 61. The memories 73 and 75 as well as the MR image memory (not shown) in the reconstruction unit 76 can be realized by way of a single memory of adequate capacity. The Fourier transformation unit 72, the composition unit 74 and the reconstruction unit 76 can be realized by way of a data processor well-suited for running a computer program according the above mentioned method.

1. A magnetic resonance imaging method for forming a sequence of images from a plurality of signals acquired by at least one receiver antenna having a spatial sensitivity profile, comprising the steps of:
calculating an activity map as a standard deviation over a series of images acquired by a reference scan,
undersampling an actual scan of k-space corresponding to an object,
transforming the resulting data to the spatial domain to form a sequence of folded preliminary images, and
resolving the ambiguity in the preliminary images resulting from the undersampled data in k-space in forming the actual images on the basis of the activity map together with the spatial sensitivity profile.

2. A magnetic resonance imaging method as claimed in claim 1, wherein in a reference scan data for the activity map and data from which the spatial sensitivity profile are acquired in an interleaved manner.

3. A magnetic resonance imaging method as claimed in claim 1, wherein the folded preliminary images are unfolded for forming the actual images also on the basis of the spatial sensitivity profiles of the receiver antennae.

4. A magnetic resonance imaging method as claimed in claim 1, wherein the reduction factor representing the degree of undersampling is an integer or non-integer number greater than 1.

5. A magnetic resonance imaging apparatus for obtaining a dynamic image from a plurality of signals comprising
means for applying a main magnetic field and magnetic gradient fields,
at least one receiver antenna for recording signals, each receiver antenna having a spatial sensitivity profile,
means for calculating an activity map as a standard deviation over a series of images acquired by a reference scan,
means for sampling the object in an actual scan of k-space by undersampling,
means for transforming the resulting data to the spatial domain to form a sequence of folded preliminary images, and
means for resolving the ambiguity in the preliminary images resulting from the undersampled data in k-space in forming the actual images on the basis of the activity map together with the spatial sensitivity profile.

6. A magnetic resonance imaging apparatus according to claim 5, wherein a body coil and one or more synergy coils are provided.

7. A magnetic resonance imaging apparatus according to claim 5, further comprising means for unfolding of the folded preliminary images for forming the actual images also on the basis of the spatial sensitivity profiles of the receiver antennae.

8. A computer readable medium containing instructions for controlling a computer system to form a dynamic image by steps comprising:
applying a main magnetic field and magnetic gradient fields,
acquiring magnetic resonance signals by at least one receiver antenna having a spatial sensitivity profile, whereas aliasing of the magnetic resonance image arises due to field inhomogenities and/or undersampling in k-space,
calculating an activity map as a standard deviation over a series of images acquired by a reference scan,
sampling the object in an actual scan of k-space by undersampling,
transforming the resulting data to the spatial domain to form a sequence of folded preliminary images, and
resolving the ambiguity in the preliminary images resulting from the undersampled data in k-space in forming the actual images on the basis of the activity map together with the spatial sensitivity profile.

9. A computer readable medium according to claim 8, the steps further comprising unfolding of the folded preliminary images for forming the actual images also on the basis of the spatial sensitivity profiles of the receiver antennae.
